(19) United States
(12) Patent Application Publication
(10) Pub. No.: US 2011/0245652 A1
(43) Pub. Date: Oct. 6, 2011

(54) IMAGING APPARATUS AND IMAGING METHOD

(75) Inventor: Takuji Oishi, Kawasaki-shi (JP)

(73) Assignee: CANON KABUSHIKI KAISHA, Tokyo (JP)

(21) Appl. No.: 13/031,726

(22) Filed: Feb. 22, 2011

(30) Foreign Application Priority Data
Mar. 31, 2010 (JP) 2010-083715

(51) Int. Cl. A61B 8/14

(52) U.S. Cl. 600/407

(57) ABSTRACT

Provided is an imaging apparatus for acquiring information on a profile of properties in a subject for enhancing an SN ratio of an image without changing a profile of intensity, by using threshold processing with an effective threshold, without reference waveform. The apparatus includes: an unit for calculating a correlation coefficient for each voxel/pixel in to obtain a profile of correlation coefficient; an unit for determining an effective threshold; an unit for judging whether or not the correlation coefficient of each voxel/pixel exceeds the effective threshold; and an unit for setting to zero or reducing each voxel/pixel whose correlation coefficient is equal to or less than the effective threshold, with respect to in vivo information on a profile of properties correlated spatially to the profile of correlation coefficient.

### BACKGROUND OF THE INVENTION

1. Field of the Invention

The present invention relates to an imaging apparatus and an imaging method. In particular, the present invention relates to an imaging apparatus and an imaging method using photoacoustic imaging or ultrasound echo imaging.

2. Description of the Related Art

In general, an imaging apparatus using an X-ray or an acoustic wave has been used in a number of fields requiring non-destructive testing such as the medical field. Image data generated using an acoustic wave (typically, ultrasound) has a drawback of a low contrast, and as a non-invasive biological information imaging method overcoming the drawback, photoacoustic tomography (PAT) that is one of optical imaging technologies has been proposed.

The photoacoustic tomography is a technology of irradiating a subject with pulsed light generated from a light source and imaging an acoustic wave generated from an in vivo material (optical absorber) that has absorbed energy of the light propagating and dispersing in the subject. According to the PAT, changes with the passage of time in acoustic wave are detected at multiple places surrounding a subject, signals thus obtained are analyzed mathematically, i.e., reconstructed, and information on a profile of properties related to in vivo optical properties are set to image data. By obtaining optical energy absorption density from an in vivo profile of initial acoustic pressure, a profile of intensity of optical properties such as an in vivo optical absorption coefficient can be obtained, and in vivo information such as the position of malignant tumor can be obtained.

The reconstruction is performed in back projection of multiple signals. This processing is described briefly below. A propagation time obtained from the positional relationship between a voxel in the reconstructed region and a detector is calculated, and signals obtained by multiple detectors are adjusted by the propagation time and added up. This result is used as an intensity of the voxel, and this process is performed with respect to all the voxels to prepare a profile of intensity.

As a procedure of enhancing the SN ratio of the profile of intensity, C.-K. Liao, M.-L. Li, and P.-C. Li, “Optoacoustic imaging with synthetic aperture focusing and coherence weighting”, OPTICS LETTERS, Vol. 29, No. 21, (2004) describes a method using a correlation coefficient obtained by digitizing the variation of multiple signals. The correlation coefficient is obtained by replacing the calculation of adding up signals with the calculation of the variation of signals in the back projection. A voxel of an image which generates a large signal has a small variation and a large correlation coefficient, whereas a voxel of a background without an image has a large variation and a small correlation coefficient. Thus, Liao et al. realize the enhancement of the SN ratio of an image by taking a product of the calculated profile of correlation coefficient and a profile of intensity.

Even in the field of ultrasound echo imaging that transmits/receives ultrasound which is an acoustic wave, a procedure of enhancing image quality is similarly performed using a correlation coefficient, although the reconstruction procedure and the calculation of a correlation coefficient are different.

Japanese Patent Application Laid-Open No. 2002-272736 regarding the enhancement of image quality using a correlation coefficient in an ultrasound echo imaging apparatus discloses a procedure in which a correlation coefficient is derived between a reference waveform previously obtained and a detected waveform when an ultrasound is transmitted/received with respect to a subject such as a living body, the derived correlation coefficient is subjected to threshold processing, image data is generated based on this, and a profile of intensity based on the generated image data is displayed.

### SUMMARY OF THE INVENTION

However, the method of enhancing image quality by taking a product of a profile of correlation coefficient and a profile of intensity as in Liao et al. has a problem that a value of a profile of intensity is changed when the product thereof with respect to the correlation coefficient is taken. Even when a maximum value of a correlation coefficient is normalized to 1, a variation occurs in the correlation coefficient to some degree due to a signal noise. Therefore, when the product is taken, a profile of intensity is changed due to the variation thereof, and further, the value is weakened. The absolute value of a profile of intensity represents initial acoustic pressure information involved in the absorption of light energy in a photoacoustic imaging apparatus, and acoustic impedance information (proportional to acoustic pressure information of a reflected acoustic wave) in the ultrasound echo imaging apparatus, respectively. Therefore, it is important that the value of a profile of intensity is not changed when quantitative evaluation of obtaining acoustic pressure information and acoustic impedance information is performed.

In order to solve such problem that a profile of intensity cannot be obtained correctly due to the influence of a profile of correlation coefficient, it is effective to provide a threshold in a correlation coefficient as in Japanese Patent Application Laid-Open No. 2002-272736, and display intensity information only on a voxel of a correlation coefficient of a threshold or more.

According to the method of Japanese Patent Application Laid-Open No. 2002-272736, a correlation coefficient is obtained from a reference waveform and a detected signal. However, the reference waveform is changed depending upon the depth due to non-linear propagation properties of ultrasound, and therefore when a correlation with a detected signal is taken in a subject, it is necessary to use a reference waveform corresponding to each depth to be detected. Thus, the procedure of Japanese Patent Application Laid-Open No. 2002-272736 involves a cumbersome operation of obtaining a reference waveform for each depth.

Further, Japanese Patent Application Laid-Open No. 2002-272736 does not refer to a threshold determining procedure, although it refers to threshold processing of extracting a portion of a threshold or more of the obtained correlation coefficient. Therefore, when a threshold is to be obtained by an ordinary method for practical use, it is necessary to calculate an effective threshold from a relationship between multiple profiles of intensity and a correlation coefficient. At this time, in order to obtain multiple profiles of intensity for calculating an effective threshold, it is necessary to obtain a signal under a changed condition, which causes a problem that processing takes a time.

In view of the above, it is an object of the present invention to provide an imaging method of enhancing an SN ratio of an image without changing a value of a profile of intensity by using threshold processing having an effective threshold determining procedure, requiring no reference waveform.

In view of the above-mentioned problem, a photoacoustic imaging apparatus according to the present invention is an imaging apparatus for acquiring in vivo information on a profile of properties from multiple signals obtained when pulsed light is incident upon a subject and an acoustic wave excited from the incident pulsed light is received by an acoustic detector. The imaging apparatus includes: a unit for deriving a profile of correlation coefficient for calculating a correlation coefficient for one of each voxel and each pixel in a detecting region from the multiple signals to obtain a profile of correlation coefficient; a threshold calculating unit for determining an effective threshold with respect to the profile of correlation coefficient; a threshold judging unit for judging whether or not the correlation coefficient of one of each voxel and each pixel exceeds the effective threshold determined in the threshold calculating unit with respect to the profile of correlation coefficient; and a threshold processing unit for setting the information on a profile of properties of one of each voxel and each pixel whose correlation coefficient is equal to or less than the effective threshold to zero or reducing the information on a profile of properties, as a result of judging by the threshold judging unit, with respect to the in vivo information on a profile of properties correlated spatially to the profile of correlation coefficient.

Further, an ultrasound echo imaging apparatus according to the present invention is an imaging apparatus for acquiring in vivo information on a profile of properties from multiple signals obtained when an acoustic wave is incident upon a subject and a reflected acoustic wave obtained by reflecting the incident acoustic wave is received by multiple acoustic detectors. The imaging apparatus includes: a unit for deriving a profile of correlation coefficient for calculating a correlation coefficient for one of each voxel and each pixel in a detecting region from the multiple signals to obtain a profile of correlation coefficient; a threshold calculating unit for determining an effective threshold with respect to the profile of correlation coefficient; a threshold judging unit for judging whether or not the correlation coefficient of one of each voxel and each pixel exceeds the effective threshold determined in the threshold calculating unit with respect to the profile of correlation coefficient; and a threshold processing unit for setting the information on a profile of properties of one of each voxel and each pixel whose correlation coefficient is equal to or less than the effective threshold to zero or reducing the information on a profile of properties, as a result of judging by the threshold judging unit, with respect to the in vivo information on a profile of properties correlated spatially to the profile of correlation coefficient.

Further, an imaging method using photoacoustic tomography according to the present invention is an imaging method of acquiring in vivo information on a profile of properties from multiple signals obtained when pulsed light is incident upon a subject and an acoustic wave excited from the incident pulsed light is received by an acoustic detector. The imaging method includes: a first step of calculating a correlation coefficient for one of each voxel and each pixel in a detecting region from the multiple signals; a second step of determining an effective threshold with respect to the profile of correlation coefficient calculated in the first step; a third step of judging whether or not a correlation coefficient of one of each voxel and each pixel exceeds the effective threshold determined in the second step with respect to the profile of correlation coefficient calculated in the first step; and a fourth step of setting the information on a profile of properties of one of each voxel and each pixel whose correlation coefficient is equal to or less than the effective threshold to zero or reducing the information on a profile of properties, as a result of judging in the third step, with respect to the in vivo information on a profile of properties correlated spatially to the profile of correlation coefficient.

Further, an imaging method using ultrasound according to the present invention is an imaging method of acquiring in vivo information on a profile of properties from multiple signals obtained when an acoustic wave is incident upon a subject and a reflected acoustic wave obtained by reflecting the incident acoustic wave is received by multiple acoustic detectors. The imaging method includes: a first step of calculating a correlation coefficient for one of each voxel and each pixel in a detecting region from the multiple signals; a second step of determining an effective threshold with respect to the profile of correlation coefficient calculated in the first step; a third step of judging whether or not a correlation coefficient of one of each voxel and each pixel exceeds the effective threshold determined in the second step with respect to the profile of correlation coefficient calculated in the first step; and a fourth step of setting the information on a profile of properties of one of each voxel and each pixel whose correlation coefficient is equal to or less than the effective threshold to zero or reducing the information on a profile of properties, as a result of judging in the third step, with respect to the in vivo information on a profile of properties correlated spatially to the profile of correlation coefficient.

The imaging apparatus according to the present invention can enhance an SN ratio of an image effectively without changing a value of a profile of intensity.

Further features of the present invention will become apparent from the following description of exemplary embodiments with reference to the attached drawings.

### BRIEF DESCRIPTION OF THE DRAWINGS

FIG. 1 is a schematic view illustrating a configuration of an apparatus according to one embodiment of the present invention.

FIG. 2 is a schematic view illustrating a flow of data processing of the apparatus according to the embodiment of the present invention.

FIG. 3 is a graph showing an example of a function used for calculating an effective threshold.

FIG. 4 is a graph showing an example of the function used for calculating an effective threshold.

FIG. 5 is a flowchart illustrating an operation of the apparatus according to the embodiment of the present invention.

FIG. 6 is a schematic view illustrating a configuration of the apparatus according to another embodiment of the present invention.

FIG. 7 is a schematic view illustrating a configuration of the apparatus according to still another embodiment of the present invention.

FIG. 8 is a schematic view illustrating a flow of data processing of the apparatus according to still another embodiment of the present invention.

### DESCRIPTION OF THE EMBODIMENTS

The present invention is applicable to any imaging apparatus that obtains subject information on a profile of properties from multiple signals obtained when an acoustic detector receives an acoustic wave propagating in the subject. As a non-limiting example of such an imaging apparatus, there are given a photoacoustic imaging apparatus and an ultrasound echo imaging apparatus. It is to be understood that while the embodiments below exemplify the case where information of an in vivo material is obtained, the applicable scope of the present invention is not limited to a living organism.

### Basic Embodiment

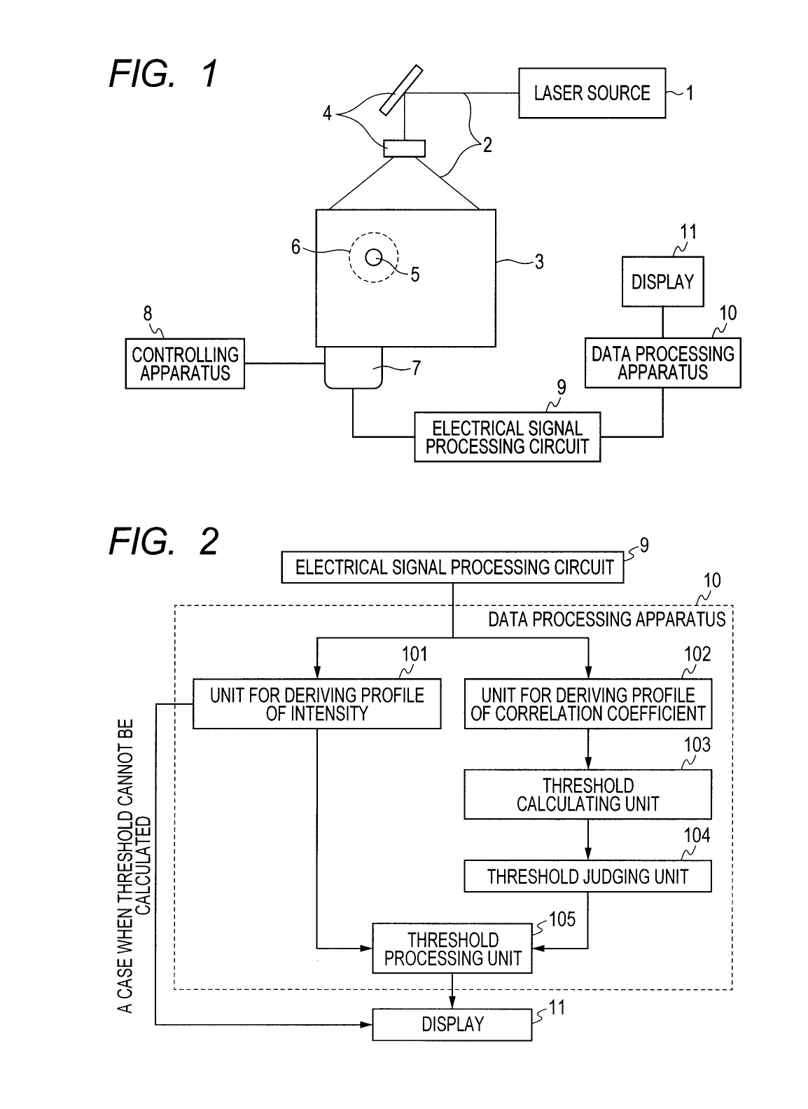

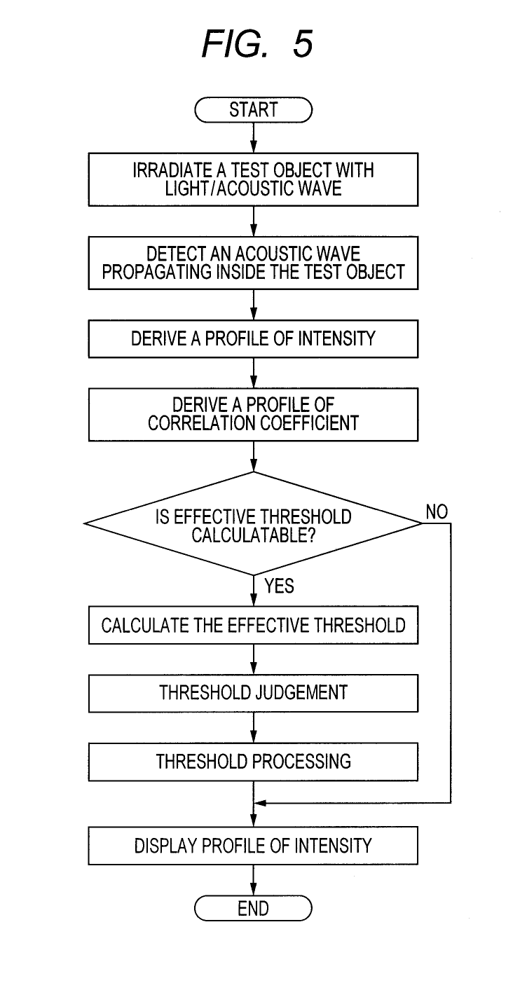

A basic embodiment of the present invention is described with reference to the drawings, using an example applied to the photoacoustic imaging apparatus (herein, photoacoustic tomography). FIG. 1 illustrates a first embodiment of biological information imaging of the present invention. Further, FIG. 5 is a flowchart thereof. Herein, a mode for carrying out the present invention is described with reference to FIG. 1 or 5.

An imaging apparatus in this embodiment includes a laser light source 1 irradiating a subject 3 with light 2, an optical device 4 such as a lens that guides the light 2 radiated from the laser light source 1 to the subject 3, an acoustic detector 7 that carries both functions of detecting an acoustic wave 6 generated by an optical absorber 5 when the optical absorber absorbs energy of the light and converting the detected acoustic wave 6 into an electric signal, a controlling apparatus 8 that causes the acoustic detector 7 to scan, an electric signal processing circuit 9 that performs amplification, digital conversion, etc. with respect to the electric signal, a data processing apparatus 10 that constructs image data regarding a profile of intensity information on optical properties that are in vivo information (subject information) on a profile of properties, and a display 11 that displays the image.

When the subject is irradiated with the pulsed light 2, the optical absorber 5 in the subject that has absorbed the incident pulsed light expands in volume due to an increase in temperature, and the acoustic wave 6 is excited to be generated. The generated acoustic wave 6 is detected by the acoustic detector 7. The acoustic detector is acoustically coupled to the subject so as to be capable of measuring the acoustic wave 6 at various places while being moved mechanically by the controlling apparatus 8. The detected electric signal is converted into a digital signal by the electric signal processing circuit 9 such as an amplifier and an analog-digital converter. Further, the image data is generated by the data processing apparatus 10 such as a personal computer (PC), and is displayed as an image on the image display 11 such as a display. In the present invention, the image data to be generated indicates subject information (information on a profile of properties such as a profile of light absorption coefficient in a living body), irrespective of whether it is two-dimensional or three-dimensional. The image data is configured in such a manner that multiple pieces of pixel data are arranged in the case where the image data is two-dimensional, and is configured in such a manner that multiple pieces of voxel data are arranged in the case where the image data is three-dimensional. In the following embodiments including this embodiment, although the case where three-dimensional image data (voxel data) is generated is described, the embodiments can be similarly applied to the case where two-dimensional image data (pixel data) is generated.

FIG. 2 illustrates internal processing of the data processing apparatus 10 carrying out the present invention. A digital signal converted by the electric signal processing circuit 9 is sent to the data processing apparatus 10, and sent to a unit 101 for deriving a profile of intensity and a unit 102 for deriving a profile of correlation coefficient inside the data processing apparatus 10. In the unit 101 for deriving a profile of intensity, multiple digital signals converted based on the acoustic wave obtained at multiple positions are subjected to filtering processing, followed by back projection, and thus, the intensities at all the voxels, i.e., a profile of intensity, in an in vivo detecting region is created. On the other hand, in the unit 102 for deriving a profile of correlation coefficient, variations in the multiple digital signals due to the acoustic waves obtained at multiple positions are digitized by Formula (1) with respect to the respective voxels, and thus, correlation coefficients at all the voxels (i.e., a spatial profile of correlation coefficient) are obtained. Herein, signal (i, t) represents a signal at a time t in an i-th detector element, and N represents the total number of detector elements. The time t is obtained considering a delay time calculated from each detection position and a positional relationship of voxels. Further, the calculated correlation coefficient may be calculated using standard deviation, variance, etc. because the variation is at least needed to be digitized.

$$\begin{matrix}[ Formula 1 ] & \\ σ = \frac{{\vert {\sum }_{i = 0}^{N - 1} Signal ( i , t ) \vert}^{2}}{N {\sum }_{i = 0}^{N - 1} {\vert Signal ( i , t ) \vert}^{2}} & ( 1 )\end{matrix}$$

The calculated profile of correlation coefficient is given to a threshold calculating unit 103 to calculate an effective threshold. A method of calculating an effective threshold is described later. Next, in a threshold judging unit 104, it is judged whether or not a correlation coefficient of a voxel exceeds an effective threshold for each voxel of a profile of correlation coefficient. In the threshold processing unit 105, regarding the in vivo information on a profile of properties (the information on a profile of properties in the subject), intensity information of a voxel spatially correlated to the voxel whose correlation coefficient is judged whether it exceeds a threshold in the profile of correlation coefficient is subjected to processing, using the judgement result and the correlation coefficient. In the case where the correlation coefficient of the voxel is more than an effective threshold in a correlation coefficient judging unit, no processing is performed with respect to intensity information of the corresponding voxel. In the case where the correlation coefficient of the voxel is below (equal to or less than) an effective threshold in the correlation coefficient judging unit, the intensity information of the corresponding voxel is set to zero. Alternatively, in the case where the correlation coefficient is below an effective threshold, the intensity information of the corresponding voxel may be decreased by a method of taking a product of the intensity information of the corresponding voxel and the correlation coefficient.

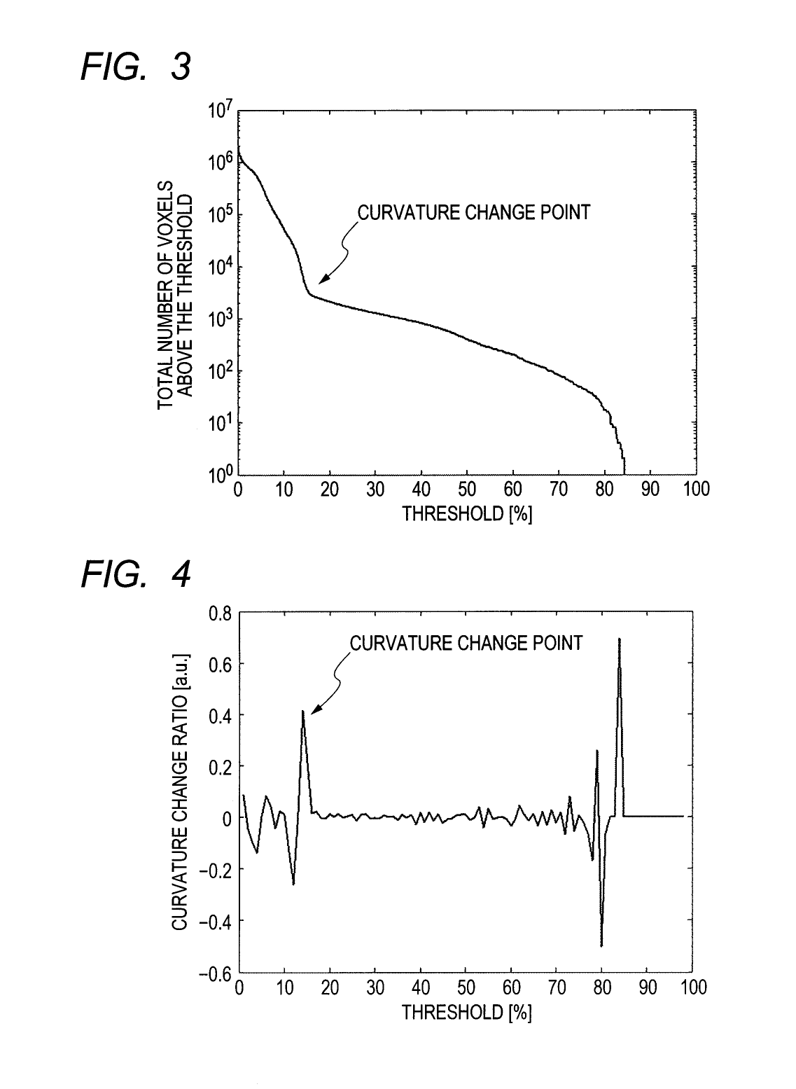

In the present invention, the intensity information of a voxel whose correlation coefficient is equal to or less than an effective threshold is set to zero or reduced, and hence an desired effective threshold is such a value that a larger number of background portions is set to be equal to or less than a threshold, while an image portion is not set to be equal to or less than a threshold. Hereinafter, an example of a method of calculating such an effective threshold is described. However, the present invention is not limited to the following method, and an effective threshold may be calculated by any method as long as the above-mentioned effective threshold can be calculated. FIG. 3 is a semilogarithmic graph showing, as a horizontal axis, a threshold set with respect to a profile of correlation coefficient obtained by processing a signal acquired by an actual photoacoustic imaging apparatus, and as a vertical axis, a logarithmic value of a total number of voxels having values of correlation coefficients equal to or more than the threshold. Herein, the value of a correlation coefficient set as a threshold when, the correlation coefficient equal to 1 is set to 100% in the horizontal axis and the correlation coefficient equal to 0 is set to 0%, is represented in terms of percentage. When the threshold is increased gradually from zero, the value of a voxel having a low correlation coefficient, i.e., the voxel in a background portion having a low correlation coefficient as a whole starts becoming less than the threshold. Note that, the correlation coefficient of the background portion is not constant and has a width due to the influence of a signal noise. Therefore, unless the threshold is increased to some degree, the entire background portion cannot become equal to or less than the threshold. On the other hand, a strong acoustic wave is generated in the image portion. Therefore, the signal varies less, and the correlation coefficient calculated in Formula (1) approaches 1. However, even in the image portion, the correlation coefficient does not become 1 completely due to the influence of a signal noise and has a width to some degree similar to the case of the back ground portion. Therefore, when the threshold is increased to some degree, the correlation coefficient in the image portion starts becoming less than the threshold. It is desired that a point at which the correlation coefficient of the image portion starts becoming less than the threshold be an effective threshold. This threshold is given at a point of a lowest threshold (point where the correlation coefficient is lowest) in multiple curvature change points in the graph of FIG. 3. In most cases, this is caused by the following: In the background portion, the correlation coefficient varies largely; however, in the image portion, the correlation coefficient is close to 1 with less variation. Thus, there is a difference in a ratio (slope in the graph of FIG. 3) between the number of respective occupying voxels and the width of correlation coefficients, which varies the slope in the function of FIG. 3. The curvature change point refers to a point where a curvature of a function changes largely to some degree, and it can be arbitrarily set to which degree a curvature of a function should change to set a curvature change point, depending upon the purpose and target of measurement. Further, the function that can be used for calculation of an effective threshold is not limited to the function of FIG. 3. For example, when a graph showing a threshold set to a correlation coefficient as a horizontal axis and the number of voxels having a correlation coefficient of the threshold as a vertical axis is used, a point having the lowest threshold among points where the number of voxels changes largely may be set to an effective threshold. Even in this case, it can be determined arbitrarily to which degree the number of voxels should be changed for determining a point to be a candidate for an effective threshold. Further, a derivative or a high-order derivative (derivative of a first order or higher) of the function of FIG. 3 may be used. Further, processing of taking a logarithmic value may be performed. FIG. 4 is a second derivative of the function of FIG. 3. Referring to FIG. 4, the curvature change point of FIG. 3 appears as a peak, and the curvature change point can be recognized easily using the high-order derivative. In FIG. 4, a curvature change point to be an effective threshold is represented as a positive peak having the smallest threshold. In the same way as in the case of using FIG. 3, even in the case of calculating an effective threshold with reference to FIG. 4, it can be arbitrarily set to which degree a peak should have to set a curvature change point, depending upon the purpose and target of measurement. In the case of creating two-dimensional image data, an effective threshold can be judged similarly based on a graph using the number of pixels instead of the number of voxels.

In the case where the ratio between the number of occupying voxels and the width of a correlation coefficient is the same between the background portion and the image portion, a slope becomes the same, and a curvature change point cannot be obtained. Thus, the procedure of the present invention cannot be used. However, such a case is extremely rare, and can be considered as a specific example. Further, in the case where an SN ratio of a signal is poor, and a calculation precision of a correlation coefficient is poor, the difference in a correlation coefficient between the background portion and the image portion becomes less. Therefore, similarly, a clear curvature change point cannot be obtained, and the procedure of the present invention cannot be used. In those cases, the SN ratio of an image cannot be enhanced by a correlation coefficient, and hence, a profile of intensity should be displayed without performing special processing.

According to the embodiment described above, in the photoacoustic imaging apparatus, only a value of a profile of intensity in the background portion can be set to zero or reduced without changing a value of a profile of intensity in an image portion, by setting an effective threshold. Further, a correlation coefficient shows variation, and is unlikely to be influenced even by a change in intensity. Therefore, even in the case where an image of high intensity and an image of low intensity are arranged, a profile of intensity of an image can be extracted with a SN ratio at a similar degree, by providing the same threshold to the correlation coefficient.

### Embodiment Applied to an Ultrasound Echo Imaging Apparatus

An embodiment of an ultrasound echo imaging apparatus using a linear array ultrasound probe (acoustic detector) is described with reference to FIG. 6. This embodiment is not limited to the linear array and can be applied to any ultrasound probe such as a convex array and a sector. However, it is preferred to use the linear array ultrasound probe because a scanning line interval is small and measurement with higher resolution can be performed.

A probe 21 is set so as to come into contact with the subject 22 such as a living body via an acoustic matching member, and an acoustic wave 24 is allowed to be incident from the probe 21. The transmitted incident acoustic wave 24 is reflected from an in vivo interface 23 where acoustic impedance is changed, such as an organ in a living body, and the probe 21 receives the reflected acoustic wave 25. The probe 21 is controlled by the controlling apparatus 26, and a signal is obtained for each scanning line in the linear array probe. The obtained reflected acoustic wave is subjected to processing such as amplification, envelope detection, and analog-digital conversion in the electric signal processing circuit 27, and is converted into a digital signal. In the data processing apparatus 28, a profile of intensity and a profile of correlation coefficient are calculated and processed to be displayed on the display 29. Internal processing of the data processing apparatus 28 is described with reference to FIG. 2. Signals of the respective scanning lines are adjusted with time, using a delay time obtained from a positional relationship between a receiving element and each voxel, and thereafter, added up with respect to all the voxels to obtain a profile of intensity (unit 101 for deriving a profile of intensity). Further, the signals of the respective scanning lines are adjusted with time, and thereafter, the variations are digitized with respect to all the voxels to obtain a profile of correlation coefficient (unit 102 for deriving a profile of correlation coefficient). The processing hereinafter is the same as that of the basic embodiment.

According to the embodiment described above, in the ultrasound echo imaging apparatus, only a profile of intensity in the background portion can be set to zero or reduced without changing a value of a profile of intensity in an image portion, by setting an effective threshold.

### Embodiment for Enhancing Precision of a Correlation Coefficient

If the precision of a correlation coefficient can be enhanced, a clear curvature change point is obtained even in the determination of an effective threshold in the basic embodiment, and a background portion and an image portion can be separated with the effective threshold. As a result, image quality can be enhanced.

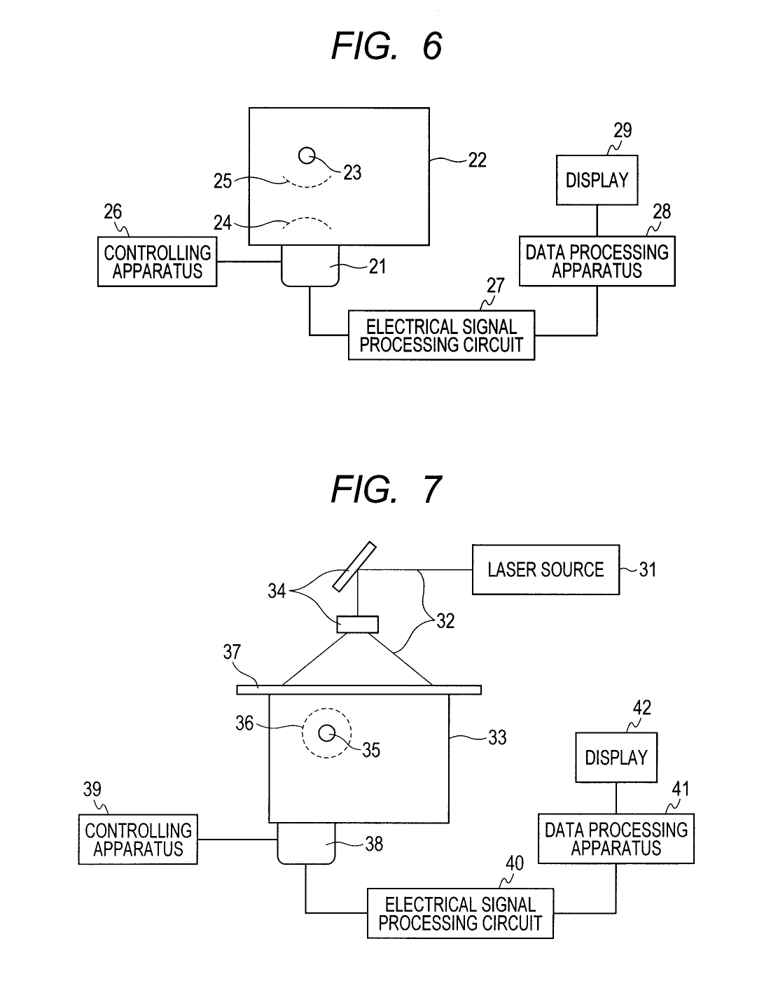

As a method of enhancing the precision of a correlation coefficient, in photoacoustic tomography, there is a method of setting a planar acoustic reflection plate at a position opposed to an acoustic detector with a subject interposed therebetween and allowing the acoustic detector to detect the acoustic wave reflected from the acoustic reflection plate. This embodiment is described with reference to FIG. 7. A subject 33 is irradiated with light 32 from a laser light source 31 by an optical device 34 such as a lens, and an acoustic wave 36 is generated in a spherical shape from an optical absorber 35 that has absorbed the irradiated light. Therefore, the acoustic wave also propagates to a side opposite to the acoustic detector 38. The acoustic wave propagating to the opposite side is reflected from the acoustic reflection plate 37, and propagates toward the acoustic detector 38 controlled by a controlling apparatus 39. As the acoustic reflection plate, it is desired to use a plate made of polycarbonate, etc. that has an acoustic impedance different from that of the subject. The acoustic wave is subjected to amplification and analog-digital conversion by an electric signal circuit 40, and processed by a data processing apparatus 41 as described later. Then, the result is displayed on a display 42.

Processing content in the data processing apparatus 41 is as follows. The acoustic wave generated from the optical absorber 35 and propagating directly to the acoustic detector is referred to as a direct wave, and the acoustic wave propagating to the acoustic detector after once being reflected from the reflection plate is referred to as a reflected wave. At this time, a profile of correlation coefficient also including a reflected wave is created and bent to be multiplied at an interface of the reflection plate. Thus, an image portion in the profile of correlation coefficient strengthens, and an intensity ratio between the background and the image, i.e., the SN ratio of the profile is enhanced. A profile of intensity and a profile of correlation coefficient by the direct wave, and a profile of intensity and a profile of correlation coefficient by the reflected wave are created respectively. Note that, the profile by the reflected wave is inverted due to reflection, and hence, the obtained profile should be inverted. By taking a product of the profile by the reflected wave and the profile by the direct wave, a profile of intensity and a profile of correlation coefficient of high precision can be obtained. The processing after the profile of intensity and the profile of correlation coefficient according to this procedure is the same as that of the basic embodiment.

According to this embodiment, the precision of the profile of intensity and the profile of correlation coefficient can be enhanced, and consequently, the SN ratio can be enhanced.

### Embodiment Using Multiple Wavelengths

An embodiment is described, which uses incident light having multiple different wavelengths with respect to the same subject in photoacoustic tomography. Herein, although an embodiment using two kinds of wavelengths is described, three or more wavelengths may be used.

The process up to the generation of a digital signal using the electric signal processing circuit 9 of FIG. 1 is the same as that of the basic embodiment. At this time, using incident light having different wavelengths, i.e., incident light having a wavelength A and a wavelength B, measurement is conducted for each incident light to obtain a digital signal.

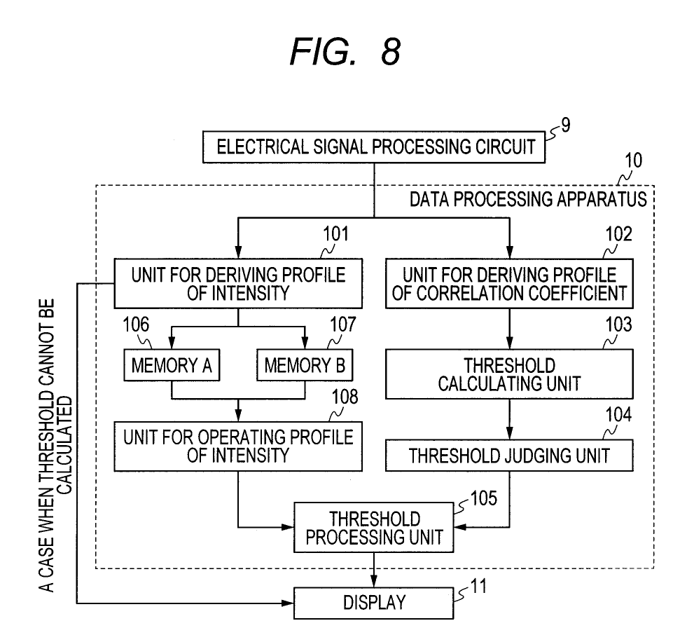

Internal processing of the data processing apparatus 10 is described with reference to FIG. 8. Digital signals obtained using the wavelength A is sent to the unit 101 for deriving a profile of intensity and the unit 102 for deriving a profile of correlation coefficient. In the unit 101 for deriving a profile of intensity, the digital signals A obtained at multiple positions are subjected to filtering, followed by back projection, to create a profile of intensity A. The profile of intensity A thus obtained is once stored in a memory A106. Next, similarly, a profile of intensity B is calculated based on digital signals obtained using the wavelength B and stored in a memory B107. Next, the profile of intensity A and the profile of intensity B stored in the memories A106 and B107 respectively are subjected to an operation of taking a ratio therebetween in a unit 108 for operating a profile of intensity, and thus, a profile of spectroscopic intensity is obtained. In the case of using at least three kinds of wavelengths, the profiles of intensity to be obtained are stored similarly in a memory C, a memory D, and so on, and a profile of spectroscopic intensity is obtained by an operation of taking a ratio of the profiles of intensity stored in the respective memories.

On the other hand, in the unit 102 for deriving a profile of correlation coefficient, the variation of any of the digital signals A or the digital signals B obtained at multiple positions is digitized to obtain a profile of correlation coefficient. Alternatively, a product of the digital signals A and the digital signals B may be used for calculating a profile of correlation coefficient. In the case of using at least three kinds of wavelengths, the variation of digital signals of any one of the wavelengths may be used, or a product of at least two kinds of digital signals selected arbitrarily from the obtained digital signals may be used. The calculated profile of correlation coefficient is given to the threshold calculating unit 103, where an effective threshold is calculated in the same way as in the basic embodiment, and in the threshold judging unit 104, it is judged whether or not the correlation coefficient of the voxel exceeds the effective threshold for each voxel. In the case where the correlation coefficient of the voxel is equal to or more than the effective threshold, no processing is performed with respect to the information on spectroscopic intensity of the corresponding voxel. In the case where the correlation coefficient of the voxel is below the effective threshold in the correlation coefficient judging unit, the value of the information on spectroscopic intensity of the corresponding voxel is set to zero or a product between the value of the information on spectroscopic intensity and the correlation coefficient is taken. The result is displayed on the display 11. Further, regarding the case where a curvature change point is not obtained, a profile of intensity is displayed as it is without performing special processing, as in the case of the basic embodiment.

According to this embodiment, information that cannot be obtained in the basic embodiment, such as a profile of spectroscopic intensity, can be obtained, and further, even in the case where some processing is performed with respect to a profile of intensity such as a profile of spectroscopic intensity, image quality can be enhanced by threshold processing using a profile of correlation coefficient.

### Another Embodiment

The present invention is not limited to a single apparatus having the above-mentioned configuration. The present invention is realized using a method of realizing the above-mentioned functions, and is also realized by the processing of supplying software (computer program) realizing those functions to a system or an apparatus via network or various storage media and allowing the system or a computer (or a CPU, an MPU, etc.) of the apparatus to read the program to execute it.

### Example

An example in which the basic embodiment is carried out is described. A base material for a subject was obtained by mixing a intralipid (soybean oil) with water so that a light scattering coefficient and an optical absorption coefficient became close to those of a human body and was molded so as to form a rectangular solid using agar. An optical absorber obtained by mixing a intralipid (soybean oil), water, and Chinese ink in a ratio of 0.08%, followed by molding the mixture into a spherical shape with agar, was placed in the subject. The subject was placed in air, and pulsed light of the order of nanoseconds having a wavelength of 1064 nm was allowed to be incident repeatedly upon the subject from one side so as to impinge on the entire surface of the subject, using an Nd:YAG laser. Although not shown in FIG. 1, an acoustic transmission plate formed of a methylpentene polymer whose acoustic impedance was close to that of a living body was set on a surface opposite to the surface upon which the pulsed light was incident and attached to the subject, and a two-dimensional array acoustic detector was attached to the subject with the acoustic transmission plate interposed therebetween. An acoustic matching member was provided between the acoustic transmission plate and the acoustic detector. Each element of the used two-dimensional array acoustic detector has a frequency band of 1 MHz±40%. The two-dimensional array acoustic detector was moved mechanically to perform light irradiation and detection of an acoustic wave at each detecting point. The interval between the respective detecting points was 6 mm, and at each detecting point, respective electric signals were obtained performing light irradiation and detection of an acoustic wave three times. The electric signals were amplified and converted into digital signals by digital-analog conversion, and an analog-digital converter used at this time had a sampling frequency of 20 MHz and a resolution of 12 bits. The digital signals at the respective detecting points were averaged, and the averaged signal was subjected to differentiation and low-pass filtering to obtain a filtered signal. The filtered signal was subjected to back projection of adjusting and adding up propagation times to the respective voxels to obtain a profile of intensity. Similarly, the propagation times to the respective voxels were adjusted regarding the filtered signal and Formula (1) was applied to obtain a profile of correlation coefficient. The profile of correlation coefficient thus obtained was examined for the relationship between the total number of the voxels having correlation coefficients of a threshold or more and the threshold to create the graph of FIG. 3. After taking a logarithmic value of this function, differentiation was performed twice, and thus, a graph of FIG. 4 was obtained. A peak appearing at about 80 to 85% of the horizontal axis of FIG. 4 is caused by discontinuous points at the same position of FIG. 3. Thus, a threshold forming the peak indicated by an arrow of FIG. 4 was determined to be an effective threshold. Next, voxels having correlation coefficients equal to an effective threshold or more in the profile of a correlation coefficient were judged and provided with tugs. Regarding the voxels provided with the tags, the profile of intensity was not changed, and regarding the voxels without the tags, the profile of intensity was set to zero. Thus, a final profile of intensity was obtained.

At this time, the ratio between the intensity value of the voxel with the largest intensity in the image portion and the average intensity value of the background portion was 140 when the profile of intensity of the voxels having correlation coefficients equal to or less than the effective threshold was not set to zero, whereas the ratio was able to be enhanced to 2400 by using the present invention. Further, the profile of intensity of the image portion was different from the original profile of intensity when the profile of intensity of the voxels having correlation coefficients equal to or less than the effective threshold was not set to zero, whereas the profile of intensity of the image portion was matched with the original profile of intensity in the present invention.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2010-083715, filed Mar. 31, 2010, which is hereby incorporated by reference herein in its entirety.

What is claimed is:

1. An imaging apparatus for acquiring in vivo information on a profile of properties from multiple signals obtained when an acoustic wave propagating in a subject is received at multiple positions by an acoustic detector, the imaging apparatus comprising:
a unit for deriving a profile of correlation coefficient for calculating a correlation coefficient for one of each voxel and each pixel in a detecting region from the multiple signals to obtain a profile of correlation coefficient;
a threshold calculating unit for determining an effective threshold, with respect to the profile of correlation coefficient;
a threshold judging unit for judging whether or not the correlation coefficient of one of each voxel and each pixel exceeds the effective threshold determined in the threshold calculating unit, with respect to the profile of correlation coefficient; and
a threshold processing unit for setting the information on a profile of properties of one of each voxel and each pixel whose correlation coefficient is equal to or less than the effective threshold to zero or reducing the information on a profile of properties, as a result of judging by the threshold judging unit, with respect to the in vivo information on a profile of properties correlated spatially to the profile of correlation coefficient.

2. An imaging apparatus according to claim 1, wherein the threshold calculating unit determines the effective threshold by obtaining a curvature change point whose threshold is the lowest in a graph showing a relationship between a threshold set to a correlation coefficient and the number of one of voxels and pixels having a value of a correlation coefficient equal to or more than the threshold, with respect to the profile of correlation coefficient.

3. An imaging apparatus according to claim 1, wherein the threshold calculating unit determines the effective threshold by obtaining a point having the lowest threshold of points where the number of one of voxels and pixels change largely in a graph showing a relationship between a threshold set to a correlation coefficient and the number of one of voxels and pixels having a correlation coefficient of the threshold, with respect to the profile of correlation coefficient.

4. An imaging apparatus according to claim 1, wherein the threshold calculating unit determines the effective threshold from a primary or higher derivative of a function representing a relationship between a threshold set to a correlation coefficient and the number of voxels having a value of a correlation coefficient equal to or more than the threshold.

5. An imaging apparatus according to claim 1,
wherein pulsed light is incident upon the subject and the acoustic wave is an acoustic wave excited from the incident pulsed light, and
wherein the unit for deriving a profile of correlation coefficient calculates a profile of correlation coefficient by multiplying profiles of correlation coefficients calculated respectively from an acoustic wave exited from the incident pulsed light and propagating directly to the acoustic detector, and an acoustic wave reflected from an acoustic reflection plate set at a position opposed to the acoustic detector with the subject interposed therebetween and propagating to the acoustic detector.

6. An imaging apparatus according to claim 1, wherein the threshold processing unit sets the corresponding information on a profile of properties to zero in a case where the correlation coefficient is equal to or less than the effective threshold, and does not process the corresponding information on a profile of properties in a case where the correlation coefficient is more than the threshold.

7. An imaging apparatus according to claim 1, wherein the threshold processing unit takes a product of a value of the corresponding information on a profile of properties and the correlation coefficient in a case where the correlation coefficient is equal to or less than the effective threshold, and does not process the corresponding information on a profile of properties in a case where the correlation coefficient is more than the threshold.

8. An imaging method of acquiring in vivo information on a profile of properties from multiple signals obtained when an acoustic wave propagating in a subject is received by an acoustic detector, the imaging method comprising:
a first step of calculating a correlation coefficient for one of each voxel and each pixel in a detecting region from the multiple signals;
a second step of determining an effective threshold, with respect to the profile of correlation coefficient calculated in the first step;
a third step of judging whether or not a correlation coefficient of one of each voxel and each pixel exceeds the effective threshold determined in the second step, with respect to the profile of correlation coefficient calculated in the first step; and
a fourth step of setting the information on a profile of properties of one of each voxel and each pixel whose correlation coefficient is equal to or less than the effective threshold to zero or reducing the information on a profile of properties, as a result of judging in the third step, with respect to the in vivo information on a profile of properties correlated spatially to the profile of correlation coefficient.

9. A program for causing a computer to carry out each step of the imaging method according to claim 8.
